(19) United States
(12) Patent Application Publication
(10) Pub. No.: US 2003/0032031 A1
(43) Pub. Date: Feb. 13, 2003

(54) Use of specific antibody titers to predict hepatic failure in people infected with hepatitis C virus

(76) Inventors: James J. Goedert, Silver Springs, MD (US); John A. Todd, Lafayette, CA (US)

Correspondence Address: REED & ASSOCIATES, 800 MENLO AVENUE, SUITE 210, MENLO PARK, CA, 94025, US

(21) Appl. No.: 10/098,857

(22) Filed: Mar. 13, 2002

Related U.S. Application Data
(63) Continuation of application No. 09/616,823, filed on Jul. 14, 2000, now abandoned.
(60) Provisional application No. 60/143,851, filed on Jul. 15, 1999.

(51) Int. Cl. C12Q 1/70; C12Q 1/68; G01N 33/53; A61K 39/29

(52) U.S. Cl. 435/6; 435/5; 435/7.1; 424/225.1

(57) ABSTRACT

A method is provided for determining the risk of hepatic failure in an individual who is infected with the hepatitis C virus (HCV). The method comprises measuring a titer of an antibody to the HCV core in a body fluid of the individual and correlating the titer to the risk of developing hepatic failure wherein the titer is inversely related to that risk. The method employs standard techniques for measuring the titer including solution and solid phase immunoassays. Kits incorporating the reagents and instructions for carrying out the methods are also provided.

### CROSS REFERENCE TO RELATED APPLICATION

This application claims priority to U.S. Provisional Application Serial No. 60/143,851, filed Jul. 15, 1999.

### TECHNICAL FIELD

This invention relates generally to methods for determining the likelihood of hepatic failure in certain individuals. More particularly, the invention relates to a novel method for determining the risk of hepatic failure in an individual who is infected with the hepatitis C virus (HCV). In addition, the invention relates to kits that allow for the facile implementation of the method.

### BACKGROUND

Hepatitis C virus (HCV), a single-stranded RNA virus of the genus Flaviviridae, was discovered in 1989 and subsequently has been shown to account for most cases of non-A non-B post-transfusion hepatitis. Choo et al. (1989) Science 244:359-362; Alter et al. (1989) N. Engl. J. Med. 321(22):1494-1500. Approximately 80 percent of patients with acute disease develop persistent HCV infection which manifests as detectable viremia with or without chronic hepatic aminotransferase elevations. Alter et al. (1993) Infect. Agents Dis. 2:155-166; Alter (1994) N. Engl. J. Med. 330(11):784-786. Those patients suffering from chronic hepatitis C over the span of 20 years have up to a 20 percent risk for developing cirrhosis. Tong et al. (1995) N. Engl. J Med. 332:1463-1466; Koretz et al. (1993) Ann. Intern. Med. 119:110-115. Once cirrhosis is established, hepatocellular carcinoma develops at a rate of one to four percent per year. Del Olmo et al. (1998) J. Cancer Res. Clin. Oncol. 124:560-564.

In the United States, most HCV infections result from shared paraphernalia for drug use, exposure to contaminated body fluids by health care workers, or transfusion of blood or blood products prior to donor screening and other safety procedures implemented in the early 1990s. Alter (1994) N. Engl. J. Med. 330(11):784-786. During the early and mid-1980s, the drug-using and blood-transfused populations also were at high risk of human immunodeficiency virus (HIV) infection. Among parenteral drug users in Baltimore, for example, 25 percent were infected with both viruses and 64 percent with HCV alone. Thomas et al. (1995) Medicine (Baltimore) 74:212-220. In addition, about half of the U.S. hemophilia population was infected with both HCV and HIV, and an additional 26 to 38 percent was infected with HCV but not HIV. Eyster et al. (1993) J. Acquir. Immune Defic. Syndr. 6:602-610. The seroprevalence rates of HCV approach 100 percent among those who required frequent infusions of plasma-derived factor VIII or factor IX concentrates. Id.

Previous reports suggested that HCV/HIV co-infected patients have a high risk of liver failure. Eyster et al. (1993) J. Acquir. Immune Defic. Syndr. 6:602-610; Telfer et al. (1994) Br. J. Haematol. 87:555-561. A significant number of HIV-negative patients infected with HCV, however, are at risk for liver failure as well. To date, there is no reliable method for screening such individuals to assess the risk of liver failure. Thus, it would be advantageous to provide a means for screening HCV-infected individuals (with and without HIV co-infection) to determine the risk for developing liver failure.

### SUMMARY OF THE INVENTION

Accordingly, it is a primary object of the invention to address the above-described needs in the art by providing a method for determining the risk of liver failure in an individual who is infected with HCV.

It is another object of the invention to provide a method for determining the risk of liver failure in an individual who is infected with HCV based on measuring a titer of an antibody to the HCV core in a body fluid of the individual and correlating the titer to the risk of developing liver failure.

It is yet another object of the invention to provide a method for determining the risk of liver failure in an individual who is infected with HCV based on measuring antigen-antibody complexes in a body fluid of the individual.

It is still another object of the invention to provide a kit for determining the risk of liver failure in an individual who is infected with HCV.

Additional objects, advantages and novel features of the invention will be set forth in part in the description which follows, and in part will become apparent to those skilled in the art upon examination of the following, or may be learned by practice of the invention.

Accordingly, in a first embodiment, a method is provided for determining the risk of liver failure in an individual who is infected with HCV, comprising the steps of measuring a titer of an antibody to the HCV core in a body fluid of the individual and correlating the titer to the risk of developing liver failure, wherein the titer is inversely related to the risk for developing liver failure.

Preferably, the invention provides that the measuring step is performed by: conducting an assay under conditions that permit formation of antigen-antibody complexes by contacting a sample of body fluid with a quantity of an antigen that binds to the antibody to the HCV core and forms an antigen-antibody complex when the antibody to the HCV core is present in the body fluid, wherein the quantity of the antigen added is sufficient to bind to substantially all antibody to the HCV core present in the sample of body fluid; and determining the quantity of antigen-antibody complexes formed in the assay. It is preferred that the assay is an enzyme immunoassay (EIA), e.g., ELISA.

It is also preferred that the assay employed in the measuring step is a solid phase immunoassay. Preferred solid phases used in the immunoassay include, for example, beads, membranes, microparticles and plates. Enzyme immunoassays comprising a solid phase are particularly preferred and include, for example, a recombinant immunoblot assay (RIBA® available from Chiron Corp., Emeryville Calif.). Recombinant immunoblot assays employ a nitrocellulose membrane as the solid phase and a recombinantly produced antigen.

In a second embodiment, a kit is provided for determining the risk of liver failure in an individual who is infected with HCV, comprising: an antigen that binds to an antibody specific for a portion of the HCV core; a substrate having the antigen attached to a surface thereon; and a set of instructions setting forth an explanation of how to measure the antibody specific for a portion of the HCV core using the antigen and the inverse correlation of a titer to the risk for developing liver failure.

In a third embodiment, a method of using the C22 antigen is provided for determining the risk of liver failure in an individual who is infected with the hepatitis C virus (HCV), comprising: the C22 antigen; measuring a titer of an antibody to the C22 antigen; and correlating the titer to the risk of developing liver failure, wherein the titer is inversely related to the risk for developing liver failure.

### BRIEF DESCRIPTION OF THE DRAWINGS

FIG. 1 provides the sequence of the first 122 amino acids of the HCV virus.

FIG. 2A shows the proportion of HCV-positive individuals developing liver failure to HIV status, as evaluated in the Example.

FIG. 2B shows the proportion of HIV- and HCV-positive individuals developing liver failure by age group, as evaluated in the Example.

FIG. 3A shows the viral load of various HCV genotypes in a sample of HCV-positive individuals, as evaluated in the Example.

FIG. 3B shows the viral load of a sample HCV-positive individuals by age group, as evaluated in the Example.

FIG. 4 shows the proportion of HCV-positive individuals developing liver failure by age group, as evaluated in the Example.

### DETAILED DESCRIPTION OF THE INVENTION

I. Definitions and Overview

Before describing the present invention in detail, it is to be understood that this invention is not limited to the specific reagents, assay formats, or the like, as such may vary. It is also to be understood that the terminology used herein is for the purpose of describing particular embodiments only and is not intended to be limiting.

It must be noted that, as used in this specification and the appended claims, the singular forms “a”, “an” and “the” include plural referents unless the context clearly dictates otherwise. Thus, for example, reference to an “antibody specific to the HCV core” includes one, two or more such antibodies, reference to an “antibody-antigen complex” includes one, two or more such complexes, and the like. In addition, as used in this specification and the appended claims, the terms “hepatic failure” and “liver failure” have identical meanings and are completely interchangeable.

In this specification and the claims that follow, the following terminology will be used in accordance with the definitions set forth below.

Unless the context clearly indicates otherwise, the term “antigen” intends a polypeptide of at least 5 amino acids, more usually at least about 8 to 10 amino acids, that defines or contains an epitope to which an antibody specific to the HCV core will bind. The antigen may consist of the entire HCV core, an HCV core segment or a derivative thereof. The HCV core comprises the coating of the virus. Thus, the antigen may comprise the entire coating, a segment of the coating or a derivative thereof, e.g., a synthetically prepared peptide with one or more substitutions or deletions of the naturally occurring amino acid sequence. In all cases, however, the antigen must contain at least one epitope to which an antibody specific to the HCV core will bind.

As used herein, a “body fluid” refers to a sample of fluid isolated from an individual, including but not limited to, lymph fluid, milk, plasma, saliva, semen, serum, spinal fluid, tears, the external sections of the skin, and the secretions of the respiratory, intestinal, and genitourinary tracts. Preferably, the body fluid is blood serum.

As used herein, “conditions that permit formation of antigen-antibody complexes” is intended to mean those conditions of time, temperature, pH and reagent concentration sufficient to allow the antibody to bind to its complementary epitope. As is well known in the art, the time, temperature, pH and concentration required for binding depend on the particular antigen, the degree of complementarity between the antibody and the antigen, and the presence of other materials in the reaction admixture. The actual conditions necessary for binding in any particular case can be readily determined by one of ordinary skill in the art.

Typical binding conditions include the use of solutions buffered to a pH from about 7 to about 8.5, and more preferably at about 7.4, temperatures from about 28° C. to about 42° C., preferably from about 30° C. to about 38° C., and most preferably at about 37° C., and a time period of from about 1 second to about 24 hours, preferably from about 7 minutes to about 16 hours, and most preferably from about 10 minutes to about 10 hours.

“Optional” or “optionally” means that the subsequently described circumstance may or may not occur, so that the description includes instances where the circumstance does occur and instances where it does not. For example, an “optional” wash step in solid phase assays includes assays where a wash step is performed and assays where a wash step is not performed.

II. The Method

The invention provides a method for determining the risk of liver failure in an individual who is infected with HCV. The method comprises measuring a titer of an antibody to the HCV core in a body fluid of the individual and correlating the titer to the risk for developing liver failure. The risk for liver failure is inversely related to the titer of antibodies specific for the HCV core.

The method requires an HCV-positive individual. Often, but not always, such an individual may also be co-infected with (HIV), hepatitis B virus (HBV) and/or other viruses. Co-infected HCV-positive individuals can also benefit from the present method.

Once established in the individual, the HCV replicates itself and invades new cells. In response, the immune system of the individual develops antibodies to various components of the virus. Generally these antibodies are IgG or IgM antibodies, but the immune system often produces other immunoglobulin varieties as well. The antibodies distribute themselves throughout the individual's body and are present in body fluids that may be extracted from the individual. Using techniques well known in the art, a sample of body fluid is withdrawn from the individual, prepared (when necessary) for analysis using known techniques and measured to determine the individual's antibody titer.

Measuring the individual's antibody titer is accomplished by employing any assay format that can detect antibodies specific for the HCV core. It is preferred that the measurement method employ an assay selected from the group consisting of centrifugation, chromatography, electrophoresis, enzyme immunoassay (EIA), immunoprecipitation, passive agglutination, recombinant immunoblot assay (RIBA®), and solid phase affinity. It is preferred, however, that the assay employed in the measuring step is a recombinant immunoblot assay. These assays are known in the art and can be adapted to measuring the titer of the antibody to the HCV core in a body fluid by those skilled in the art using only routine experimentation. Another preferred assay is an enzyme immunoassay (EIA).

In order to identify specific antibodies to the HCV core, the assay must employ an antigen that contains the entire HCV core, a portion of the HCV core or a derivative thereof. Preparation, of antigens for use with the present method is discussed infra.

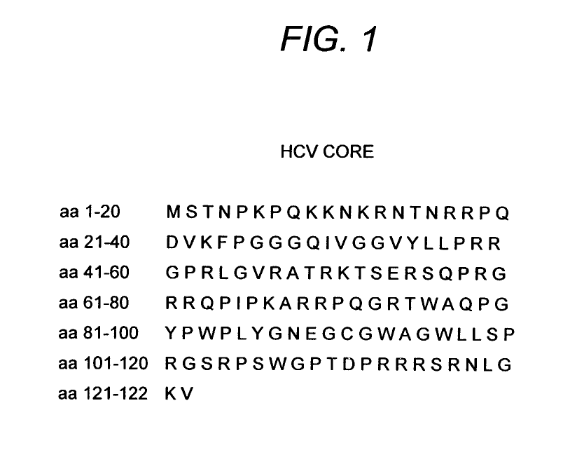

A common feature to all of the assays useful herein is that an antigen is employed having at least one epitope from the HCV core. The core of the HCV virus comprises amino acids 1-120 of FIG. 1. The HCV core antigen may be in the form of a polypeptide composed entirely of the HCV core amino acid sequence, a portion of the HCV core amino acid sequence or a derivative thereof. The core contains many epitopes, i.e., binding sites for a specific antibody. Thus, when only a portion of the HCV core sequence is employed as the antigen, that portion must comprise at least one epitope. It is preferred that the antigen comprises the epitopes present in the HCV core sequence known as “C22.” Alternatively, this sequence can be identified as the “C22 antigen.”

Once the epitope or epitopes have been selected, the antigen must be prepared. The antigen is preferably prepared by employing recombinant technology via techniques well known in the art. Specifically, recombinant techniques such as constructing DNA encoding the desired antigen, cloning the DNA into an expression vector, transforming a host cell, e.g., bacteria, yeast, or mammalian cell, and expressing the DNA to produce the desired antigen are well known. Advantageously, the recombinantly produced HCV antigen may be produced as a fusion protein, i.e., as a protein formed by expression of a hybrid gene made by combining two or more gene sequences. Thus, for example, the appropriate DNA gene sequence of the desired protein is inserted into an expression vector of an existing gene encoding a protein such as β-galactosidase or ubiquitin using techniques well known in the art. For example, the DNA sequences coding for the two genes (i.e., the natural gene and the gene encoding the desired antigen) is introduced to the DNA of a host cell by, for example, a plasmid vector conferring resistance to an antibiotic. Once the cells have multiplied, those host cells harboring the desired gene sequences are selected, in this case, by application of the appropriate antibiotic to the culture medium. Thereafter, the selected cells are cloned and the desired protein, i.e., the fusion protein, is recovered from the cells using conventional techniques. For example, the fusion protein may be recovered by lysing the cells, separating the proteins, e.g., by size exclusion chromatography and collecting the fusion protein. This and other recombinant techniques serve to increase the level of expression and/or increase the water solubility of the antigen. In addition, these techniques allow for the fusion protein to display multiple epitopes (either identical or different) within the same molecule.

The epitopes in the fusion proteins, however, must only be epitopes from the HCV core. For instance, a continuous fragment of DNA encoding repeated epitopes contained within the C22 antigen may be constructed, cloned into an expression vector and used to express a fusion protein having multiple epitopes from the C22 sequence. In a similar manner, fusion proteins of C22 antigen and an alternative HCV core antigen may be employed. As previously indicated, the C22 antigen is defined by the sequence of amino acids 1-122 of FIG. 1.

Alternatively, the antigens may be synthetically produced. Synthetic production of antigens generally employs techniques of standard solid phase peptide synthesis well known in the art. In such a method, the synthesis of peptides is sequentially carried out by incorporating the desired amino acid residues one at a time onto a growing peptide chain according to the general principles of solid phase synthesis as described, for example, by Merrifield (1963) J. Amer. Chem. Soc. 85:2149-2154. Common to chemical syntheses of peptides is the protection of reactive side chain groups of the various amino acid moieties with suitable protecting groups which will prevent a chemical reaction from occurring at that site until the protecting group is ultimately removed. It is also well known to protect the a-amino group on an amino acid while that entity reacts at the carboxyl group, followed by the selective removal of the a-amino protecting group to allow a subsequent reaction to take place at that site. Examples of suitable a-amino and side chain protecting groups are well known in the art.

Additionally, HCV core antigens can be obtained from the virus itself. For example, the virus may be cleaved using proteolytic enzymes and/or conditions sufficient to break the virus apart. The desired HCV core segments are then separated and recovered using, for example, centrifugation or size-exclusion chromatography.

The HCV core antigens may be employed in either a solution phase or solid phase immunoassay. Examples of both are well known in the art and discussed below. In solid phase immunoassays, the HCV core antigens are immobilized on a solid phase. Preferably, the antigen is bound to a solid phase selected from the group consisting of a beads, microparticles, membranes and plates. The solid phase is made from any substance suitable for immobilizing the antigen and includes, for example, nitrocellulose (e.g., in membranes), polyvinyl chloride (e.g., in sheets or microtiter wells), polystyrene latex (e.g., in beads or microtiter plates), polyvinylidine fluoride (e.g., in microtiter plates), and polystyrene (e.g., in beads). Methods for covalently or noncovalently binding the antigens to a solid phase are well known in the art. For example, the antigen will be covalently attached to a solid phase if the antigen was synthesized used standard solid phase protein synthesis. Alternatively, noncovalent interactions such as hydrogen bonding or Van der Waals forces can bind the antigen when the antigen is placed in contact with the solid phase. The antigens may be bound throughout the surface of the solid phase or may be distributed in a pattern, e.g., in bands, to facilitate detection of antigen-antibody binding.

In solution phase (or homogenous) assays, detection of the antibody is performed in a single tube without the need to separate antigen-antibody complexes from unbound antigens and antibodies. For example, an antigen containing both a fluorescent label and a quenching agent is used to detect the presence the antibody. The quencher has the ability to quench the fluorescence emission of the fluorescent label. (In this case, the fluorescent label is not detectable until its spatial relationship to the quenching agent has been altered, for example by release of the quenching agent from the antigen when binding occurs). Thus, prior to binding, the dual fluorophore/quencher labeled antigen does not emit fluorescence. Subsequent to binding, the quencher is released from the antigen thereby allowing fluorescence to be detected by the eye or automated fluorescence measurement.

Once prepared, the antigen is added to or contacted with a sample of body fluid from an HCV-positive individual. This step is performed under conditions that permit the antigen to bind to any such antibody present in the body fluid. Typically, the assay is performed at a temperature of from about 28° C. to about 42° C., preferably from about 30° C. to about 38° C., and most preferably at about 37° C. In addition, the assay is performed at a pH of about 7 to about 8.5, preferably at pH 7.4. Furthermore, antigen-antibody complex formation is allowed to proceed for a time sufficient to substantially bind all HCV core-specific antibodies to antigens. It is preferred, however, that antigen-antibody complex formation is allowed to proceed for about 10 minutes to about 10 hours after the antigen is added to the body fluid.

In solid phase assay formats, the solid phase is optionally washed in order to remove any reagents, unbound antibodies and similar moieties that may affect the specificity or sensitivity of the assay. Generally, however, it is preferred that one or more wash steps is performed during and/or following the solid phase assay.

Once antigen-antibody complex formation has taken place, it is then necessary to detect and measure the quantity of complexes formed. Typically, detection and measurement of complex formation is accomplished by use of a second antibody that is labeled and capable of binding to an HCV antibody. Suitable labels include, for example, chemiluminescent, colorimetric, enzymatic, fluorescent and radioactive moieties. Methods of amplifying the signals from the complex are also known and include, for example, use of biotin and avidin, and enzyme labeling, i.e., ELISA. Direct or indirect fluorescent assays can be employed. Measuring the signal can be accomplished using the unaided eye, a microscope or a measuring instrument such as a densitometer. Densitometers are particularly preferred for reading the bands on an assay strip.

Alternatively, antigen-antibody complex formulation is detected and measured in immunoprecipitation or agglutination assays (employing a second antibody that can bind to an HCV antibody) by detecting a network that precipitates from the solution or suspension. This network forms a visible layer or film of precipitate. If no anti-HCV antibody is present in the test specimen, no visible precipitate is formed.

Enzyme immunoassays, e.g., ELISA assays, and other immunoassays using fluorescent and chemiluminescent labels are particular preferred for conducting the measuring step of the assay of the invention. Another preferred assay for the measuring step is a recombinant immunoblot assay (RIBA® available from Chiron Corp., Emeryville Calif.). This assay employs a nitrocellulose strip having antigens, e.g., C22 antigen, a SOD band, and IgG control bands. The test is very sensitive for detecting HCV antibodies and is conducted in a manner similar to a Western blot assay.

The reagents and tools necessary to carry out the method of the invention can be conveniently packaged into a kit. The kit is used for determining the risk of liver failure in an individual who is infected with HCV and comprises: an antigen that binds to an antibody specific for a portion of the HCV core; and a set of instructions setting forth an explanation of how to measure the antibody specific for a portion of the HCV core using the antigen and the inverse correlation of a titer to the risk for developing liver failure. Preferably, the kit further comprises a substrate, having the antigen attached to a surface thereof. It is also preferred that the substrate is nitrocellulose. The kit may also contain the antigen in solution, for conducting a solution phase assay. The kit may also contain reagents for binding the antigen to the substrate, a control antibody, a labeled antibody (when the assay format so requires), and signal generating reagents (e.g., enzyme substrate) if the label does not generate a direct signal.

III. Utility

Not all individuals infected with the HCV virus will develop liver failure. The method and kits of the invention, however, are effective for identifying HCV-positive individuals who are at a higher risk for liver failure. For those individuals at higher risk for liver failure, the liver failure will occur within 30 years, and potentially within 15 years. It has been found that those HCV-positive individuals who develop a relatively high titer of antibodies to the HCV core have a lower risk for developing liver failure. Conversely, those HCV-positive individuals who have a relatively low titer of antibodies to the HCV core have a higher risk for developing liver failure.

For example, it has been found that a weak antibody titer against the HCV core was associated with a 37 percent chance of liver failure fifteen years later while a strong antibody titer was associated with a 15% chance of liver failure. Antibody titers that constitute “weak” or “strong” responses can be determined experimentally by one of ordinary skill or as set forth in the Example.

HCV attacks the parenchyma of the liver causing a wide range of pathologies. Clinically, liver failure may present as: hepatic encephalopathy (due to the liver failing to clear ammonia); jaundice (due to failed clearance of bilirubin); and bleeding (due to failed synthesis of clotting factors). Unfortunately, the treatment for individuals suffering from liver failure is often only palliative in nature. Consequently, the present method is extremely useful in that it provides a means for determining which individuals are at greater risk for developing liver failure so that immediate precautions can be taken to minimize the progression to this life-threatening disease. Such precautions include, for example, refraining from alcohol and hepatoxic drugs, administering drugs to treat hepatitis C infection, e.g., interferon, and decreasing the risk of contracting other pathogens, e.g., HIV, known to increase the risk of liver failure.

It is to be understood that while the invention has been described in conjunction with the preferred specific embodiments thereof, that the foregoing description as well as the example that follows are intended to illustrate and not limit the scope of the invention. Other aspects, advantages and modifications within the scope of the invention will be apparent to those skilled in the art to which the invention pertains.

All publications mentioned herein, both supra and infra, are hereby incorporated by reference. All ranges identified herein, both supra and infra, are inclusive.

### Experimental

The following example is put forth so as to provide those of ordinary skill in the art with a complete disclosure and description of invention disclosed and claimed herein. Efforts have been made to ensure accuracy with respect to numbers (e.g., amounts, temperature, etc.) but some errors and deviation should be accounted for. Unless indicated otherwise, temperature is in ° C. and pressure is at or near atmospheric pressure at sea level.

Unless otherwise indicated, all materials and reagents were obtained commercially (e.g., from Aldrich, Sigma and ICN) and used without further purification.

### EXAMPLE 1

Methods

A prospective cohort study was performed to determine risk factors for the acquired immunodeficiency syndrome (AIDS) and related conditions among all registered patients with hemophilia and other coagulation disorders at twelve comprehensive hemophilia centers in the United States and four in Europe. At approximately annual intervals, each subject underwent a standardized physical examination (including a question on the number of alcoholic drinks per week), medical record review and phlebotomy. HCV antibody status of all subjects was determined with a commercially available second- or third-generation enzyme immunoassay, with most reactive samples confirmed by recombinant immunoblot assay (HCV RIBA® 2.0 or 3.0, Chiron Corp., Emeryville Calif.). A stratified random sample of 20 HIV-positive and 20 HIV-negative subjects in each of eight age groups was selected, most of whom had sufficient sera or plasma for determining HCV level, genotype and serotype.

HCV plasma level was determined with branched-DNA technology (Quantiplex HCV RNA 2.0, Chiron Corp., Emeryville Calif.) with a lower limit of sensitivity of 200,000 (5.3 log₁₀ copies/mL). HCV serotype was determined with the RIBA® Serotyping SIA (Chiron Corp., Emeryville Calif.). Individual RIBA® bands were scored on an 11-point gray scale from zero (pure white) to ten (pure black) using an external standard, with less than 8 on this scale (less than 3+ on a conventional visual scale) considered weak. For genotyping, HCV RNA was reverse transcribed and amplified by the polymerase chain reaction (PCR) using nested biotinylated primers to the highly conserved 5′ non-coding region. Selected samples also were amplified using a commercially available HCV RNA assay (Amplicor® HCV, Roche Diagnostic Systems, Inc., Branchburg N.J.). Genotyping was performed on the labeled amplicons using a commercially available line probe assay (LiPA, version 1.0, Immunogenetics, Zwijndrecht, Belgium). Samples with no PCR product for the LiPA are referred to as PCR-negative; those with a typical band patterns are referred to as genotype-unspecified. All samples had been stored at −70° C., and most had never been previously thawed. The three assays were performed in separate laboratories blinded to each other's results and to the background data. HCV plasma level was transformed to log₁₀ for all analyses.

After initial inspection of the data for homogeneity, the 16 strata were reduced to age quintiles (in May 1982: less than nine, 9 to 16, 17 to 23, 24 to 32, and more than 32 years) for HIV-positive and -negative subjects. Frequency distributions across these 10 strata were calculated for samples with multiple HCV genotypes, those with one of the major genotypes (HCV-1, -2, -3, and -4), and those with no detectable viral bands (n=48) or unspecified bands (n=17). Frequencies were also calculated for the definable HCV serotypes (1, 2, 3, ⅓) and those with undefinable serotype bands (n=10). The agreement beyond chance between genotype and serotype for HCV 1, HCV 2, and HCV 3 was determined with the kappa (κ) statistic. Mean [±2 standard errors (SE)] HCV levels (log₁₀ copies/mL) were compared among groups by analysis of variance.

Liver failure was defined as the earliest occurrence of esophageal varices, hepatic encephalopathy, or persistent ascites, excluding non-hepatic causes of these conditions, or at death, if attributed to liver failure. Validation of these cases was performed by independent reabstraction of all medical records and a six percent random sample of the remaining HCV seropositive subjects in the cohort. Potential contributory causes, such as medications or chronic active hepatitis B virus infection, were not excluded. The Kaplan-Meier product-limit method was used to estimate liver failure incidence [±1 standard error (SE)] from the median MHCS HIV seroconversion date (May 29, 1982) or the subject's birth date, if later. For all HCV seropositive subjects and in a case-cohort design using the random sample (Epicure, HiroSoft International Corp., Seattle Wash.), proportional hazard modeling was used to determine the relative hazard [±95 percent confidence interval (CI)] of hepatic failure by HCV genotype, HCV plasma level, HIV status, age, and other variables. All data were censored at 16 years (May 28, 1998).

Results

Of 2056 hemophilic subjects in the MHCS, 1194 (58 percent) were seropositive for HIV and HCV (“co-infected”), 624 (30 percent) were seropositive only for HCV, 20 (one percent) were seropositive only for HIV, and 218 (11 percent) were negative for both viruses.

One hundred thirty-seven of the HCV-positive subjects in the MHCS have developed hepatic failure, defined as hepatic death (including one confirmed and one suspected hepatocellular carcinoma), hepatic encephalopathy, esophageal varices, or persistent ascites without other identifiable cause. Hepatic failure developed in ten (1.6 percent) of the 624-HIV-negative subjects compared to 127 (10.6 percent) of the 1194 HIV-positive subjects. Two of the HIV-positive cases of hepatic failure occurred prior to the starting date (May 1982) and were excluded, leaving 1192 HIV-positive subjects for prospective analysis. Seventeen subjects (14 HIV-positive and three HIV-negative) were treated with interferon-a before hepatic failure, as were 14 subjects (13 HIV-positive) who have not had hepatic failure.

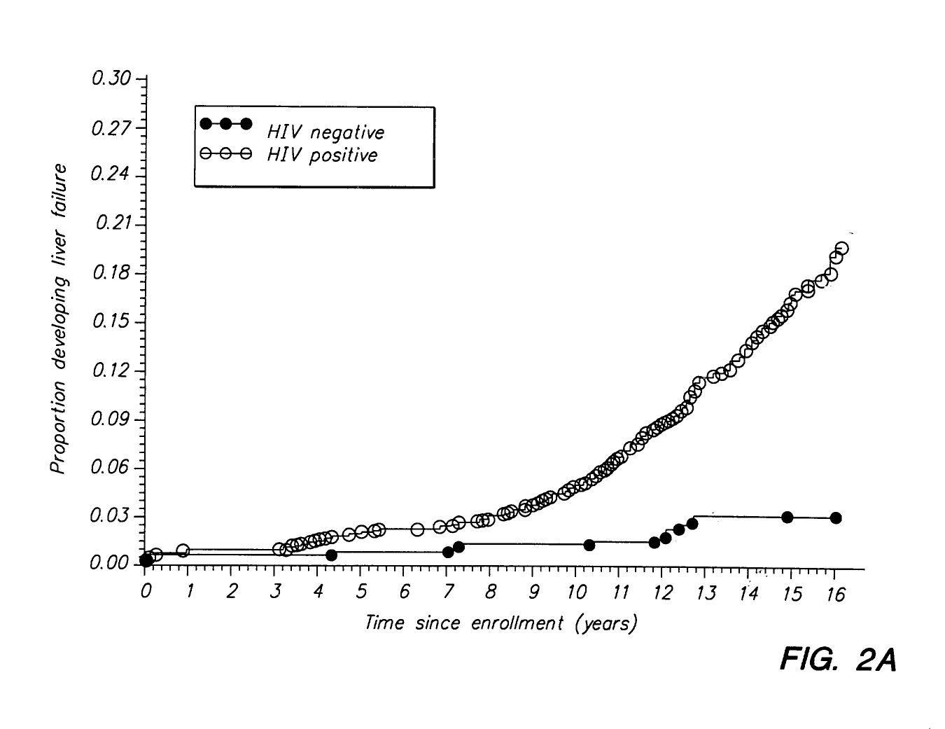

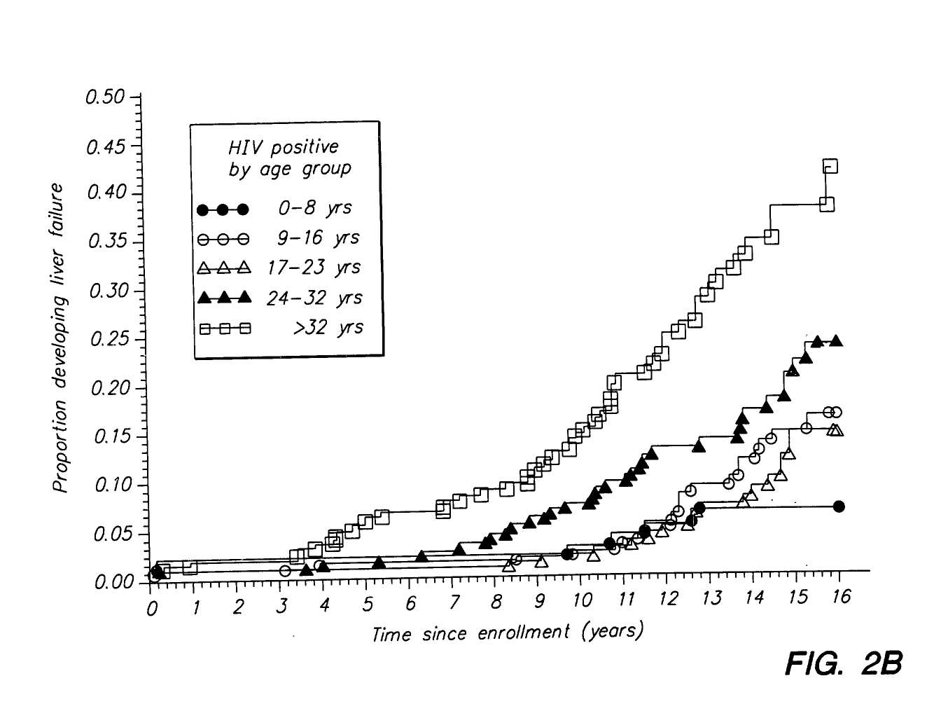

By product-limit estimation at 16 years of follow-up, hepatic failure had occurred in 20.1 (±1.9 SE) percent of the 1192 subjects who were co-infected with HIV compared to 2.7 (±0.9 SE) percent of the 624 subjects who were not infected with HIV (P<0.0001, FIG. 2A). Risk of hepatic failure was very high for the oldest subjects. Among the HIV-positive subjects, 16-year cumulative incidence of hepatic failure was 41.8 (±6.5 SE) percent in the oldest quintile (enrollment after age 32) compared to 23.6(±4.1 SE) percent in the second quintile (age 24 to 32, P=0.002). Hepatic failure was lower still in the third, fourth, and youngest (enrollment under age 9) quintiles, with incidence rates of 14.4 percent, 16.2 percent, and 6.4 percent, respectively (P<0.05 for each compared to age 24 to 32, FIG. 2B). Among the HIV-negative subjects, seven of the 10 hepatic failure cases occurred in the oldest quintile (cumulative incidence 8.6 versus 1.2 percent among younger subjects, P=0.0006). Available data were insufficient to clearly distinguish whether the association was related to older age at HCV infection, longer duration of infection, or both.

By proportional hazards modeling, hepatic failure risk was greatly increased with HIV co-infection [relative hazard 7.9 (95 percent CI 4.2, 15.2)] and older age [relative hazard 1.6 (95 percent CI 1.0, 2.5) for ages 17 to 32; relative hazard 5.0 (95 percent CI 3.2, 7.9) after age 32]. Older age appeared to be more adverse for HIV-negative than HIV-positive subjects, but the confidence intervals overlapped substantially.

Alcohol consumption as reported in three levels (none, less than eight, eight or more drinks per week) was directly related to hepatic failure among the HIV-positive subjects [relative hazard 1.3 (95 percent CI 1.02, 1.7)] and the HIV-negative subjects [relative hazard 2.3 (95 percent CI 0.96, 5.6)]. Adjustment for age attenuated this association with alcohol in the HIV-negative subjects [relative hazard 1.4 (95 percent CI 0.5, 3.6)] but not in the HIV-positive subjects [relative hazard 1.3 (95 percent CI 0.96, 1.7)].

Available serologic and vaccination data were sufficient to clearly define hepatitis B virus status for 616 subjects without HIV, among whom the 16-year cumulative incidence was unrelated to hepatitis B status (P=0.61, data not presented). With HCV and HIV co-infection, 16-year hepatic failure incidence rates were 40.5 (±10.6 SE) percent among 104 subjects with chronic hepatitis B surface antigenemia, 20.2 (±2.1 SE) percent among 985 subjects with resolved hepatitis B infection, and 3.2 (±2.3 SE) percent among 91 subjects never infected with hepatitis B virus (P=0.003). With adjustment for age, the high risk with chronic antigenemia and low risk with no hepatitis B infection were no longer significant (P=0.24 and 0.07, respectively).

The stratified random sample of 310 subjects selected for HCV genotyping, serotyping, and plasma level testing was representative of and did not differ from the experience of the MHCS population, as determined by prevalence severe hemophilia A (P=0.80); cumulative 16-year survival rates among the HIV-positive subjects (P=0.44) and HIV-negative subjects (P=0.47); and, among the HIV-positive subjects, hepatic failure incidence rates (P=0.51) and associations with age group (data not presented).

One hundred seventy-three (56 percent) of the subjects had HCV genotype 1, with nearly equal numbers of subtypes 1a and Ib. Thirty-two subjects had genotype 2, 30 had genotype 3, and three had genotype 4. Seven subjects (two percent) had multiple genotypes detected; 17 had genotypes that could not be specified; and 48 (15 percent) were PCR-negative. Two hundred forty-eight (80 percent) of the subjects had HCV serotype 1, compared to only 16 and 24 subjects with, serotypes 2 and 3, respectively. Nine subjects had faint or absent serotype bands, and for 13 subjects the assay could not distinguish serotypes 1 and 3. The level of agreement between HCV genotypes and serotypes was low for HCV 1 (K=0.25) and HCV 2 (κ=0.21) and good for HCV 3 (κ=0.44).

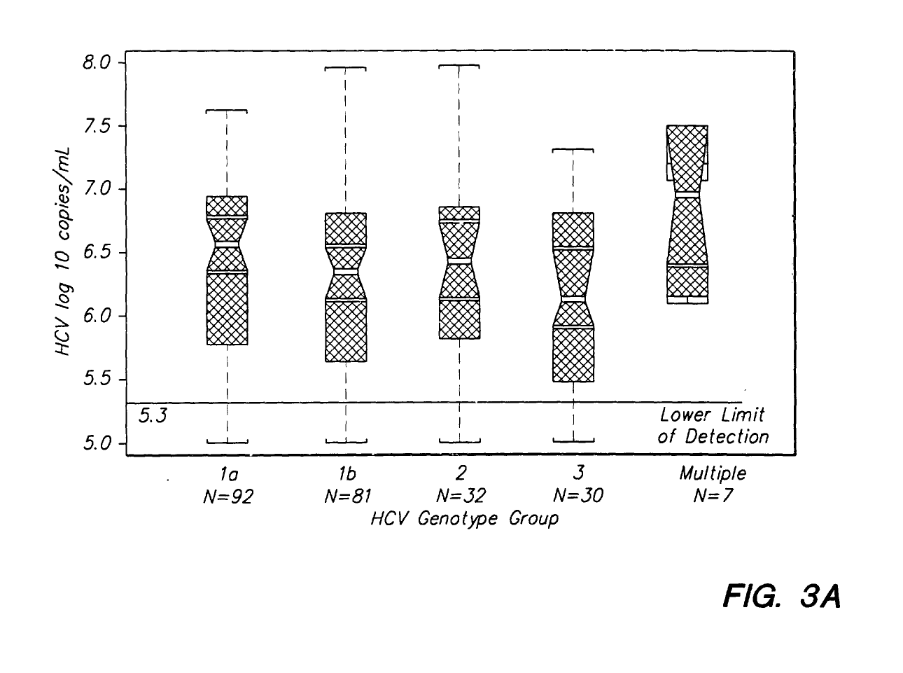

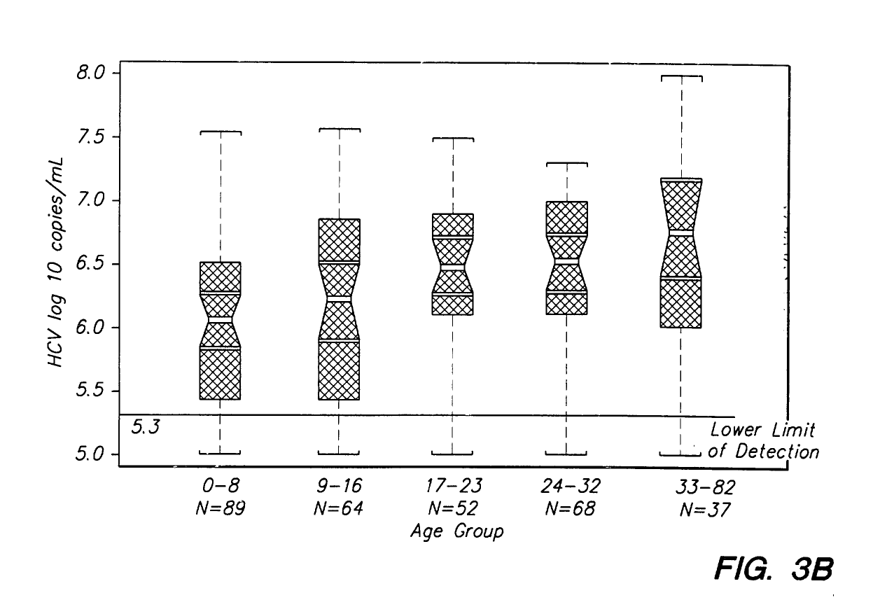

Among HIV-positive compared to HIV-negative subjects, the mean HCV plasma level was significantly higher (6.2±0.1 vs. 5.9±0.1 log₁₀ copies/mL, P=0.0001) and increased significantly more over a mean of 4.9 years (0.42±0.05 vs. 0.16±0.04 log₁₀ copies/mL, P=0.0002). Viral levels were similar in subjects with severe hemophilia A (6.1±0.1 log₁₀ copies/mL), mild or moderate hemophilia A (6.1±0.1 log₁₀ copies/mL), factor VIII inhibitors (6.1±0.1 log₁₀ copies/mL), or vonWillebrand's disease (5.9±0.3 log₁₀ copies/mL); but they were significantly lower (5.8±0.1 log₁₀ copies/mL) among subjects with hemophilia B. The HCV level was undetectable in nearly all of the PCR-negative and genotype-unspecified subjects. Among PCR-positive subjects, the HCV level was similar with HCV types 1a, 1b and 2 (6.3 log₁₀ copies/mL) but appeared to be lower with genotype 3 (6.1 log₁₀ copies/mL) and higher among the seven subjects with multiple genotypes (6.6 log₁₀ copies/mL, FIG. 3A, shaded areas representing error bars). The HCV level was similar among subjects over age 16, but it was significantly lower in the younger subjects (FIG. 3B, shaded areas representing error bars). Among HCV PCR-positive subjects, a multivariate model with genotype 1; HIV-negativity, and age less than 17 as the referent group, the HCV plasma level was independently and significantly lower with HCV genotype 3 (P=0.05) and higher with HIV-positivity (P=0.03) and older age (P<0.004). Hemophilia B, added to this model, was unrelated to HCV level (P=0.43).

In the case-cohort analysis of HIV-positive subjects, risk of hepatic failure was unrelated to HCV plasma level among all subjects (P_{trend}=0.87) and subgroups with or without HCV genotype 1 (P_{trend}=0.99 and 0.24, respectively). Risk also was unrelated to HCV genotype and serotype (P=0.18 and 0.53, respectively), although the risk appeared higher with genotype 1 than with genotype 2 or 3 (P=0.06). The intensity of the individual serotype bands revealed a higher risk of hepatic failure, with weaker antibody reactivity against HCV core-1 [relative hazard 0.86 (95 percent CI 0.77, 0.96) per unit on 11-point scale] or core-2 [relative hazard 0.88 (95 percent CI 0.78, 0.998)]. In multivariate models (not presented) significant inverse associations persisted with core-1 and CD4⁺ lymphocyte levels but not with core-2 or the three NS4 bands.

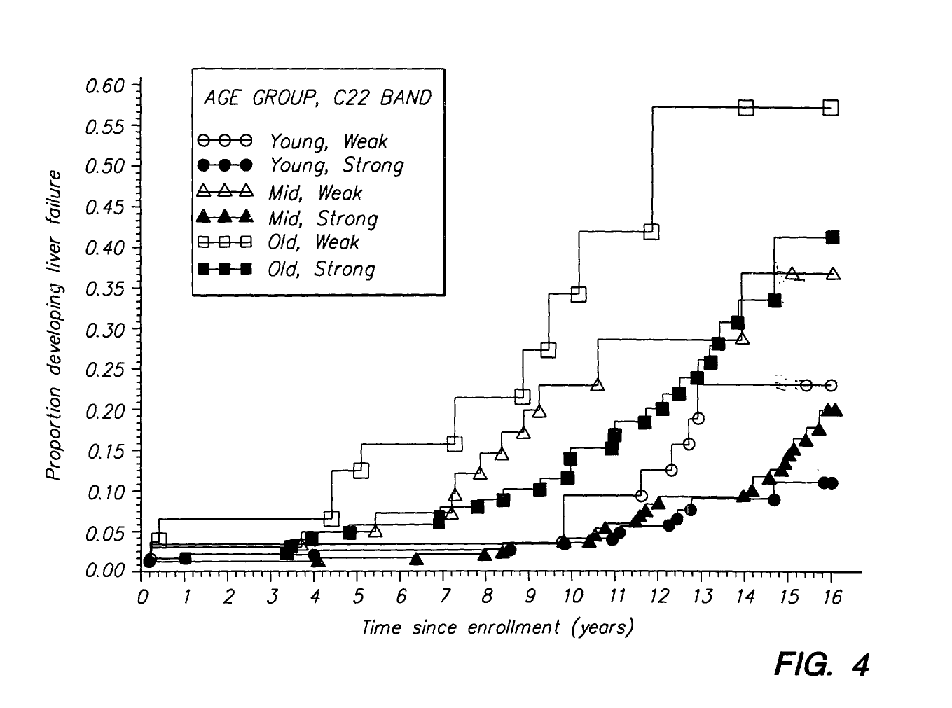

Given these results with the serotype bands, a similar analysis was performed on the bands of the RIBA® that confirmed HCV seropositivity in 747 HIV-positive subjects. Hepatic failure incidence was unrelated to strength of the c100, c5-1-1, c33c, and NS5 RIBA® bands (P>0.10, data not shown); but it was substantially elevated with weak reactivity against the C22 band. Among HIV-positive subjects, estimated 16-year hepatic failure incidence ranged from 10 percent for young subjects with strong anti-C22 reactivity to 55 percent for old subjects with weak anti-C22 reactivity (FIG. 4).

CD4⁺ and CD8⁺ lymphocyte counts had been performed near the time of the RIBA® sample in 684 HIV-positive subjects. CD4⁺ counts in the lower two quintiles (below 259 cells/μL) were associated with a significantly higher incidence of hepatic failure (P=0.0003), although risk did not differ between the two lowest nor among the three highest quintiles (P>0.71). Risk was similarly increased in the lowest quintile of CD8⁺ counts (below 307 CD8⁺ cells/μL, P=0.0008), although this association did not persist with adjustment for other variables.

HCV levels changed over a mean of 4.9 years, increasing 0.48 log₁₀ copies/mL in 44 cases of hepatic failure compared to 0.41 log₁₀ copies/mL in 242 subjects who have not had hepatic failure (P=0.60). Increases in viral levels also were noted with low CD4⁺ lymphocytes (0.50 vs 0.38 log₁₀ copies/mL, P=0.27) or low anti-C22 HCV antibodies (0.51 vs 0.27 log₁₀ copies/mL, P=0.12), but these were not statistically significant.

In the final multivariate proportional hazards model, hepatic failure risk was low for the 47 subjects never infected with hepatitis B virus (relative hazard 0.1, 95 percent CI 0.02, 1.0), was increased 2.4-fold (95 percent CI 1.5, 3.8) with a low CD4⁺ lymphocyte count, was increased 2.0-fold (95 percent CI 1.2, 3.2) with weak anti-C22 reactivity, and was strongly related to older age.

Discussion

Fifty-eight percent of the subjects in the cohort were infected with HCV and HIV, and an additional 30 percent were infected with HCV without HIV. The risk of hepatic failure was markedly increased with older age and co-infection with HIV, reaching 42 percent during 16 years of follow-up after age 32. In the multivariate analysis controlling for age and hepatitis B status, the risk of hepatic failure with HCV and HIV co-infection was increased 2.4-fold with a low CD4⁺ lymphocyte count and two-fold with a weak antibody response against the HCV C22 core protein.

These results point to a profound effect of impaired immunity on the development of HCV-related hepatic failure. Impaired CD4⁺ T-cell responses against HCV core protein was associated with chronic hepatitis C. While not wishing to be bound by theory, it is postulated that CD4⁺ T-cell responses to HCV peptides are impaired in HIV infection, yet HCV-specific cytotoxic T lymphocyte (CTL) activity may be preserved.

Weak humoral immunity against HCV core proteins was associated with a high risk of hepatic failure. Several studies have noted that the majority of patients with HIV co-infection lack or lose HCV antibody reactivity against the C100, C5-1-1, C33c, and NS5 RIBA® proteins but generally not against the C22 protein. It is not know if anti-C22 reactivity declined over time, only that it was relatively weak in subjects who progressed to hepatic failure. Although it is possible that strong anti-core antibodies are neutralizing or protective, weak antibodies may merely reflect antigen-antibody complexing, dysfunctional immune responses, or even non-immunologic pathways leading to fibrosis and ultimately hepatic failure.

Hepatic failure risk, although not related to HCV plasma level, was slightly increased with HCV genotype 1. Genotype 1 also has been associated with a higher risk of AIDS and death in HCV/HIV co-infected hemophilic patients. Sabin et al. (1997) J. Infect. Dis. 175: 164-168. More than half of the subjects were infected with HCV genotype 1.

It is likely that hepatotoxic drugs and perhaps infections increased the risk of hepatic failure. Poynard and colleagues reported that progressive hepatic fibrosis was directly related to the duration of HCV infection, older age at infection, and heavy alcohol consumption. Poynard et al. (1997) Lancet 342:825-832. The present study revealed that, with or without HIV co-infection, drinking alcohol was weakly associated with the risk of hepatic failure.

Hepatitis B virus infection is a highly prevalent, classical cause of cirrhosis. The risk of hepatic failure was elevated with chronic hepatitis B in the univariate analysis, but it is likely that this association merely reflected older age rather than contributing to the disease. In contrast, multivariate analysis revealed a significantly reduced risk for the few subjects who were never infected with hepatitis B, suggesting impaired hepatic function after resolved hepatitis B infection. Hepatic failure risk was not increased with hepatitis G virus infection in this cohort.

Thus, in subjects with HCV and HIV co-infection, the incidence of hepatic failure was unrelated to HCV level in plasma, marginally increased with HCV genotype 1 and alcohol consumption, and substantially increased with older age, a low CD4⁺ lymphocyte count and a weak antibody response against HCV core proteins.

What is claimed is:

1. A method for determining the risk of liver failure in an individual who is infected with the hepatitis C virus, comprising:
measuring a titer of an antibody to the HCV core in a body fluid of the individual; and
correlating the titer to the risk of developing liver failure,
wherein the titer is inversely related to the risk for developing liver failure.

2. The method of claim 1, wherein the body fluid is blood serum.

3. The method of claim 1, wherein the measuring step is performed by:
conducting an assay under conditions that permit formation of antigen-antibody complexes by contacting a sample of body fluid with a quantity of an antigen that binds to the antibody to the HCV core and forms an antigen-antibody complex when the antibody to the HCV core is present in the body fluid, wherein the quantity of the antigen added is an amount sufficient to bind to substantially all antibody to the HCV core in the sample; and
determining the titer by measuring the quantity of antigen-antibody complexes formed in the assay.

4. The method of claim 3, wherein the antigen is recombinantly produced.

5. The method of claim 3, wherein the antigen is synthetically produced.

6. The method of claim 3, wherein the antigen comprises C22 antigen.

7. The method of claim 3, wherein the assay is conducted at a temperature of from about 28° C. to about 42° C.

8. The method of claim 3, wherein antigen-antibody complex formation is allowed to proceed for about 10 minutes to about 10 hours after the antigen is added to the body fluid.

9. The method of claim 3, wherein the assay is a solid phase immunoassay.

10. The method of claim 9, wherein the antigen is bound to a solid phase selected from the group consisting of beads, microparticles, membranes and plates.

11. The method of claim 10, wherein the solid phase is a nitrocellulose membrane.

12. The method of claim 3, wherein the assay is a solution phase immunoassay.

13. The method of claim 1, wherein the measuring step is performed using an assay selected from the group consisting of centrifugation, chromatography, electrophoresis, enzyme immunoassay, immunoprecipitation, passive agglutination, recombinant immunoblot assay, and solid phase affinity.

14. The method of claim 13, wherein the measuring step is performed using an enzyme immunoassay.

15. The method of claim 13, wherein the measuring step is performed using a recombinant immunoblot assay.

16. The method of claim 1, wherein the antibody is an IgG or IgM antibody.

17. The method of claim 1, wherein the liver failure occurs within 30 years.

18. A kit for determining the risk of liver failure in an individual who is infected with the hepatitis C virus (HCV), comprising:
an antigen that binds to an antibody specific for a portion of the HCV core; and
a set of instructions setting forth an explanation of how to measure the antibody specific for a portion of the HCV core using the antigen and the inverse correlation of a titer to the risk for developing liver failure.

19. The kit of claim 18, further comprising a substrate having the antigen attached to a surface thereof.

20. The kit of claim 19, wherein the substrate is nitrocellulose.

21. The kit of claim 18, wherein the antigen is in solution.

22. The kit of claim 18, wherein the antigen comprises C22 antigen.

23. A method of using the C22 antigen for determining the risk of liver failure in an individual who is infected with the hepatitis C virus, comprising:
the C22 antigen;
measuring a titer of an antibody to the C22 antigen; and
correlating the titer to the risk of developing liver failure,
wherein the titer is inversely related to the risk for developing liver failure.
